(19) United States
(12) Patent Application Publication
(10) Pub. No.: US 2006/0088474 A1
(43) Pub. Date: Apr. 27, 2006

(54) Endohedral metallofullerene contrast agents

(76) Inventors: Harry C Dorn, Blacksburg, VA (US); Erick B Iezzi, Jeannette, PA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/509,874

(22) PCT Filed: Apr. 2, 2003
(86) PCT No.: PCT/US03/10137

§ 371 Date: Sep. 28, 2005

Related U.S. Application Data
(60) Provisional application No. 60/368,960, filed on Apr. 2, 2002.

(51) Int. Cl. A61K 49/00; C07F 5/00; A61K 49/10; A61K 49/04

(52) U.S. Cl. 424/9.36; 534/11; 534/15; 977/726

(57) ABSTRACT

An image contrast agent for creating an image of a portion of a body is disclosed. The contrast agent can provide contrast for two different types of imaging techniques, such as x-ray imaging and magnetic resonance imaging. The contrast agent includes an endohedral metallofullerene compound with an atom encapsulated in the fullerene cage for imaging based on one type of technique, and a second atom encapsulated in the fullerene cage for imaging based on a second type of technique. Alternatively, the contrast agent may include a first endohedral metallofullerene compound having a one type of contrast imaging characteristics, and a second endohedral metallofullerene compound having a second type of contrast imaging characteristics.

### FIELD OF THE INVENTION

The present invention relates to contrast agents for use in imaging a body. More particularly, the present invention relates to contrast agents that provide for different types of imaging using endohedral metallofullerene compounds.

### BACKGROUND OF THE INVENTION

U.S. Pat. No. 6,303,760, herein incorporated by reference in its entirety, describes a family of endohedral metallofullerenes where a trimetallic nitride is encapsulated in a fullerene cage. The endohedral metallofullerenes have the general formula A₃₋ₙXₙN@Cm (n=0-3) where A is a metal, X is a second trivalent metal, n is an integer from 0 to 3, and m is an even integer from about 60 to about 200. The metals A and X may be an element selected from the group consisting of a rare earth element and a group IIIB element and may be the same or different. These trimetallic nitride endohedral metallofullerenes are produced by introducing nitrogen gas into a Krätschmer-Huffman generator during vaporization of packed graphite rods containing corresponding metal oxides, known as the trimetallic nitride template (TNT) process.

### SUMMARY OF THE INVENTION

The present invention includes a contrast agent based on a trimetallic nitride endohedral metallofullerene compound having a first atom with a first imaging contrast property and a second atom with a second imaging contrast property, wherein the first atom and the second atom are encapsulated within the fullerene cage of the endohedral metallofullerene.

The present invention also includes a contrast agent having the formula Lu₃N@Cₘ where m is an even integer from about 60 to about 200.

Still further, the invention includes a contrast agent that includes a trimetallic nitride endohedral metallofullerene compound having a first atom with a first imaging contrast property encapsulated within a fullerene cage of the endohedral metallofullerene, and a second trimetallic nitride endohedral metallofullerene compound having a second atom with a second imaging contrast property encapsulated within a fullerene cage of the endohedral metallofullerene.

The present invention also includes a contrast imaging method. The method includes administering an effective amount of a contrast agent to a subject, where the contrast agent comprises a trimetallic endohedral metallofullerene compound having a first atom with an imaging contrast property encapsulated in the fullerene cage of the endohedral metallofullerene. The method also includes applying an imaging technique to the subject, wherein the imaging technique provides an image of a portion of a subject based on the imaging contrast property of the administered contrast agent.

### DETAILED DESCRIPTION OF EMBODIMENTS OF THE INVENTION

Contrast agents are used to absorb radiation and provide an imaging contrast of the human anatomy. These contrast agents typically contain atoms with high atomic numbers and their dense electronic environment absorbs X-ray radiation. Some common atoms used in contrast agents include iodine, boron, and barium. To produce a better contrast, it is desirable to use heavier elements. A difficulty is that many of the heavier elements that would be useful as a contrast agent may be toxic to the human body. Further, it would be useful to provide a single contrast agent that allows for the multiple imaging techniques, such as x-ray imaging and magnetic resonance imaging.

The present invention is directed to family of contrast agents that provide for the imaging of a subject using different imaging techniques. The contrast agents of the present invention allow for more than one imaging technique to be used based on a single contrast agent. For example, and as will be discussed in detail below, one contrast agent can be used to create both an x-ray image and a magnetic resonance image.

In a accordance with an embodiment of the present invention, a contrast agent includes a trimetallic nitride endohedral metallofullerene compound having a first atom with a first imaging contrast property and a second atom with a second imaging contrast property encapsulated within the fullerene cage of the endohedral metallofullerene.

The imaging contrast properties for the atoms in the contrast agent include, but are not limited to, x-ray imaging properties useful for x-ray imaging and computerized axial tomography (CAT) imaging, and magnetic resonance imaging (MRI) properties useful for magnetic resonance imaging. Elements useful for the first and second atoms include rare earth and group IIIB elements. Preferably, the first atom and second atoms may each be selected from the group consisting of lutetium, yttrium, erbium, europium, holmium, gadolinium, terbium, dysprosium, and depleted uranium. All of these elements would be useful in x-ray imaging techniques. Elements useful for magnetic resonance imaging are preferably lutetium, gadolinium, terbium, dysprosium, holmium, and erbium. In certain embodiments, the first and second atoms should provide different imaging properties. In further embodiments, the first and second atoms may be the same provided that they provide for use in different imaging techniques. For example, lutetium may be used for x-ray imaging as well as magnetic resonance imaging. In this case, lutetium may be used for the first and second atoms.

In certain embodiments lutetium may be used as the first atom, and the second atom may be selected from the group consisting of lutetium, yttrium, erbium, europium, holmium, gadolinium, terbium, dysprosium, and depleted uranium.

Any fullerene cage known to one of skill in the art may be used to encapsulate the atoms. Preferably, the fullerene cages include, but are not limited to a C₆₈ cage, C₇₈ cage, and C₈₀ cage. A preferred embodiment utilizes a C₈₀ fullerene cage.

The trimetallic nitride endohedral metallofullerene compound may have the formula A₃₋ₙXₙN@Cₘ, where A is the first atom having a first imaging contrast property and X is the second atom having a second imaging contrast property, n can range from 0 to 3, and m can range from about 60 to about 200. The first atoms and second atoms have the same criteria as that discussed above. Certain embodiments for the contrast agent include, but are not limited to, Lu₃N@C₈₀, Gd₂LuN@C₈₀, GdLu₂N@C₈₀, ErLu₂N@C₈₀, Er₂LuN@C₈₀, HoLu₂N@C₈₀, and Ho₂LuN@C₈₀.

As used herein, “endohedral” refers to the encapsulation of atoms inside a fullerene cage network. Accepted symbols for elements and subscripts to denote numbers of elements are used herein. Further, all elements to the right of an @ symbol are associated with the exterior of the fullerene cage network, while all elements listed to the left are contained within the fullerene cage network. Under this notation, Lu₃N@C₈₀ indicates that the Lu₃N trimetallic nitride is situated within a C₈₀ fullerene cage.

The embodiments discussed above are directed to a single endohedral metallofullerene having two different types of imaging properties. An accordance with another embodiment of the present invention, a contrast agent may include a mixture of endohedral metallofullerene compounds, where different endohedral metallofullerene compounds provide for different imaging properties. For example, an embodiment of the present invention includes a trimetallic nitride endohedral metallofullerene compound having a first atom with a first imaging contrast property encapsulated within a fullerene cage of the endohedral metallofullerene, and a second trimetallic nitride endohedral metallofullerene compound having a second atom with a second imaging contrast property encapsulated within a fullerene cage of the endohedral metallofullerene. The first atom and second atom have the same characteristics and imaging properties, and include the same elements as those discussed above. Likewise the fullerene cage includes the fullerene cages discussed above.

The trimetallic nitride endohedral metallofullerene compounds used in the contrast agent can also be functionalized on the exterior of the fullerene cage (“exohedral”). U.S. patent application Ser. No. 10/244,747, entitled “Endohedral Metallofullerene Derivatives,” herein incorporated by reference in its entirety describes the functionalization of endohedral metallofullerenes. The functionalization allows for changes in solubility in different solvent systems. The endohedral metallofullerene can be made more hydrophobic or hydrophilic depending on the functional group added to the exterior of the fullerene cage. Exohedral functionalization of the carbon cage can provide for improved bio-distribution. This functionalization involves one or more appendage groups attached to the carbon cage. Functionalizing the endohedral metallofullerenes can take place by various reactions, including, but not limited to, hydroxylation, Diels-Alder reactions, Bingel-Hirsch reaction, and other similar reaction mechanisms.

The present invention also includes a contrast imaging method. The method includes administering an effective amount of a contrast agent to a subject, wherein the contrast agent comprises a trimetallic endohedral metallofullerene compound having a first atom with an imaging contrast property encapsulated in the fullerene cage of the endohedral metallofullerene, and applying an imaging technique to the subject. The imaging technique provides an image of a portion of a subject based on the imaging contrast property of the administered contrast agent. The first atom, second atom, and fullerene cage may include the characteristics discussed above with respect to the embodiments for the contrast agent.

The method also includes a contrast agent that includes a second atom having a second imaging contrast property. The second atom may be encapsulated in the fullerene cage with the first atom or as part of a second endohedral metallofullerene compound as discussed above.

The effective amount of a contrast agent will vary depending on the subject and the specific contrast agent being used and requires an amount to form a image based on the technique being used. The imaging techniques may include, but are not limited to, x-ray imaging, computerized axial tomography, or magnetic resonance imaging.

Once the contrast agent is administered to the subject, the contrasting agent moves through the body. After a sufficient amount of time, the desired imaging technique is performed. The method of applying the desired imaging technique will vary depending the imaging technique and are well known by those skilled in the art.

By encapsulating the high atomic number metal atom within a carbon structure, the high atomic number metal atom cannot readily escape the structure; thus reducing the potential for toxic exposure of the high atomic number metal atom to a human. Further, by providing different imaging contrast properties in the same contrast agent, different imaging techniques can be performed on the subject with have to provide the subject with a separate and different agent.

While the invention has been described in terms of its preferred embodiments, those skilled in the art will recognize that the invention can be practiced with modification within the spirit and scope of the appended claims.

What is claimed is:

1. A contrast agent, comprising:
a trimetallic nitride endohedral metallofullerene compound having a first atom with a first imaging contrast property and a second atom with a second imaging contrast property, wherein the first atom and the second atom are encapsulated within the fullerene cage of the endohedral metallofullerene.

2. The contrast agent of claim 1, wherein the first imaging contrast property and the second imaging contrast property are different.

3. The contrast agent of claim 1, wherein the first atom is lutetium.

4. The contrast agent of claim 1, wherein the first atom is lutetium and the second atom is a rare earth or group IIIB element.

5. The contrast agent of claim 1, wherein the first atom is lutetium and the second atom is yttrium, erbium, europium, holmium, gadolinium, terbium, dysprosium, or depleted uranium.

6. The contrast agent of claim 1, wherein the first atom is lutetium, yttrium, erbium, europium, holmium, gadolinium, terbium, dysprosium, or depleted uranium; and wherein the second atom is lutetium, yttrium, erbium, europium, holmium, gadolinium, terbium, dysprosium, or depleted uranium.

7. The contrast agent of claim 1, wherein the first imaging contrast property is an x-ray absorption property for use in forming an image, and wherein the second imaging contrast property is a magnetic resonance imaging property.

8. The contrast agent of claim 1, wherein the fullerene cage is C₆₈, C₇₉, or C₈₀.

9. The contrast agent of claim 1, wherein the fullerene cage is C₈₀.

10. A contrast agent, comprising a compound having the formula:
Lu₃N@Cₘ
wherein m is an even integer from about 60 to about 200.

11. A contrast agent, comprising:
a trimetallic nitride endohedral metallofullerene compound having a first atom with a first imaging contrast property encapsulated within a fullerene cage of the endohedral metallofullerene; and
a second trimetallic nitride endohedral metallofullerene compound having a second atom with a second imaging contrast property encapsulated within a fullerene cage of the endohedral metallofullerene.

12. The contrast agent of claim 11, wherein the first imaging contrast property and the second imaging contrast property are different.

13. The contrast agent of claim 11, wherein the first atom is lutetium.

14. The contrast agent of claim 11, wherein the first atom is lutetium and the second atom is a rare earth or group IIIB element.

15. The contrast agent of claim 11, wherein the first atom is lutetium and the second atom is yttrium, erbium, europium, holmium, gadolinium, terbium, dysprosium, or depleted uranium.

16. The contrast agent of claim 11, wherein the first atom is lutetium, yttrium, erbium, europium, holmium, gadolinium, terbium, dysprosium, or depleted uranium; and wherein the second atom is lutetium, yttrium, erbium, europium, holmium, gadolinium, terbium, dysprosium, or depleted uranium.

17. The contrast agent of claim 11, wherein the first imaging contrast property is an x-ray absorption property for use in forming an image, and wherein and the second imaging contrast property is a magnetic resonance imaging property.

18. The contrast agent of claim 11, wherein the fullerene cage is C₆₈, C₇₈, or C₈₀.

19. The contrast agent of claim 11, wherein the fullerene cage is C₈₀.

20. A contrast imaging method, comprising the steps of:
administering an effective amount of a contrast agent to a subject, wherein the contrast agent comprises a trimetallic endohedral metallofullerene compound having a first atom with an imaging contrast property encapsulated in the fullerene cage of the endohedral metallofullerene; and
applying an imaging technique to the subject, wherein the imaging technique provides an image of a portion of a subject based on the imaging contrast property of the administered contrast agent.

21. The method of claim 20, wherein the imaging technique X-ray imaging, computerized axial tomography, or magnetic resonance imaging.

22. The method of claim 20, wherein the contrast agent further comprises:
a second atom with a second imaging contrast property, wherein the first atom and the second atom are encapsulated within the fullerene cage of the endohedral metallofullerene.

23. The method of claim 22, wherein the first imaging contrast property and the second imaging contrast property are different.

24. The method of claim 20, wherein the first atom is lutetium.

25. The method of claim 22, wherein the first atom is lutetium and the second atom is a rare earth or group IIIB element.

26. The method of claim 22, wherein the first atom is lutetium and the second atom is yttrium, erbium, europium, holmium, gadolinium, terbium, dysprosium, or depleted uranium.

27. The method of claim 22, wherein the first atom is lutetium, yttrium, erbium, europium, holmium, gadolinium, terbium, dysprosium, or depleted uranium; and wherein the second atom is lutetium, yttrium, erbium, europium, holmium, gadolinium, terbium, dysprosium, or depleted uranium.

28. The method of claim 22, wherein the first imaging contrast property is an x-ray absorption property for use in forming an image, and wherein and the second imaging contrast property is a magnetic resonance imaging property.

29. The method of claim 20, wherein the contrast agent further comprises:
a second trimetallic nitride endohedral metallofullerene compound having a second atom with a second imaging contrast property encapsulated within a fullerene cage of the endohedral metallofullerene.

30. The method of claim 29, wherein the first imaging contrast property and the second imaging contrast property are different.

31. The method of claim 29, wherein the first atom is lutetium, yttrium, erbium, europium, holmium, gadolinium, terbium, dysprosium, or depleted uranium; and wherein the second atom is lutetium, yttrium, erbium, europium, holmium, gadolinium, terbium, dysprosium, or depleted uranium.

32. The method of claim 29, wherein the first imaging contrast property is an x-ray absorption property for use in forming an image, and wherein and the second imaging contrast property is a magnetic resonance imaging property.
